(19) United States
(12) Patent Application Publication
(10) Pub. No.: US 2025/0002923 A1
(43) Pub. Date: Jan. 2, 2025

(54) Use of constitutively active variants of growth factor receptors as selection markers for the generation of stable producer cell lines

(71) Applicant: CEVEC Pharmaceuticals GmbH, Köln (DE)

(72) Inventors: Nicole Faust, Köln (DE); Silke Wissing, Köln (DE); Nikola Strempel, Köln (DE)

(21) Appl. No.: 18/769,083

(22) Filed: Jul. 10, 2024

Related U.S. Application Data
(63) Continuation of application No. 16/636,622, filed on Feb. 4, 2020, now Pat. No. 12,060,561.

(30) Foreign Application Priority Data
Aug. 8, 2017 (EP) 17001356.9

(51) Int. Cl. C12N 15/62; C07K 14/705; C07K 14/71; C07K 19/00; C12N 5/07; C12N 5/071; C12N 5/10; C12N 5/16; C12N 15/09; C12N 15/113; C12N 15/63; C12N 15/85

(52) U.S. Cl. CPC C12N 15/62; C07K 14/70517; C07K 14/71; C07K 19/00; C12N 5/06; C12N 5/0625; C12N 5/10; C12N 5/16; C12N 15/09; C12N 15/1138; C12N 15/63; C12N 15/85; C07K 2319/00; C12N 2501/105

(57) ABSTRACT

The present invention relates to methods for the selection of a stable producer cell line expressing one or more protein(s) of interest (POIs), comprising the stable transfection of cells with a gene encoding a constitutively active variant of a growth factor receptor, respective methods for the expression of one or more POIs in such stable producer cell lines, and uses of a constitutively active variant of a growth factor receptor as selection marker in the cultivation of cells.

This application is a continuation of U.S. application Ser. No. 16/636,622, filed Feb. 4, 2020, which claims the priority benefit of PCT/EP2018/070829, filed on Aug. 1, 2018, which claims the benefit of European Application No. EP17001356.9, filed on Aug. 8, 2017, which are incorporated herein by reference in their entireties.

The instant application contains a Sequence Listing which has been submitted electronically in ST.26 XML format and is hereby incorporated by reference in its entirety. Said ST.26 XML copy, created on Jul. 10, 2024, is named 34428_0821_SL.xml and is 7,000 bytes in size.

The present invention relates to methods for the selection of a stable producer cell line expressing one or more protein(s) of interest (POIs), comprising the stable transfection of cells with a gene encoding a constitutively active variant of a growth factor receptor, respective methods for the expression of one or more POIs in such stable producer cell lines, and uses of a constitutively active variant of a growth factor receptor as selection marker in the cultivation of cells.

Selection markers are essential tools for the generation of stable producer cell lines, i.e., cell lines that stably express one or more POIs such as e.g. biopharmaceuticals. Usually, these selection markers are antibiotic resistance genes which are encoded on the expression vector, and which are stably integrated in the target genome (e.g. by transfection) together with the gene(s) of interest (GOIs). Cultivation of the transfected cells in the presence of the respective antibiotic selects for producer cells with integrated expression vector.

Commonly used antibiotic-based selection markers for mammalian expression systems include e.g. Blasticidin S resistance genes from Bacillus cereusor Aspergillus terreus, G418 (Geneticin) resistance gene from transposon Tn5 (neo), Puromycin resistance gene from Streptomyces spp., Hygromycin B resistance gene from Escherichia coli, and Zeocin resistance gene from Streptoalloteichus hindustanus.

Drawbacks of antibiotic resistance-based selection markers include the fact that only a very limited set of selection markers is available. Further, there are issues with instable producer clones, especially when expressing very large, multimeric proteins, wherein recombinant expression of the target protein is silenced over time resulting in low titers during production. To prevent silencing, the cells can be cultured in the presence of the respective antibiotic selection agent. However, the large scale production of POIs in the presence of antibiotics is not desirable as this requires extensive removal and analytics steps after production.

Thus, there is an urgent need for new selection markers, e.g. in case that several expression constructs need to be stably integrated, allowing for enhanced stability of cell lines and ease of use. Ideally, such new selection markers support the sustained expression of the POI in the absence of selection agents.

Accordingly, the technical problem underlying the present invention is to provide respective selection markers and methods using the same.

The solution to the above technical problem is achieved by the embodiments characterized in the claims.

In particular, in a first aspect, the present invention relates to a method for the selection of a stable producer cell line expressing one or more protein(s) of interest (POIs), comprising the steps of:
(a) providing a cell line,
(b) concurrently stably transfecting said cell line with
a gene encoding a constitutively active variant of a growth factor receptor, wherein growth of said cell line in the absence of expression of said constitutively active variant of a growth factor receptor is dependent on the growth factor that is recognized by said growth factor receptor, and
(ii) one or more gene(s) of interest (GOIs) encoding said POIs, and
(c) cultivating said cell line in a cell culture medium either not containing the growth factor that is recognized by said growth factor receptor or containing said growth factor at concentrations which are too low to support growth of the cells in the absence of expression of said constitutively active variant of a growth factor receptor.

Usually, growth factor receptors are activated by binding of the respective ligand, i.e., the respective growth factor (e.g. IGF-1, IFG-2 or insulin for IGF-1R), and subsequent dimerization leading to downstream phosphorylation events and activation of growth-promoting and anti-apoptotic pathways, including MAP-Kinase and NFkappaB pathways. These growth factors are present in serum and need to be supplemented when cultivating cells which are dependent on these factors in serum-free growth medium. Cells which show a growth response to IGF-1 and insulin include CAP, CHO, BHK, HEK 293, Vero, PER.C6®, MDCK cells, hybridoma cells, and fibroblasts. Cultivation of such cells usually requires supplementation of growth factors which increases the costs of respective production processes. The present invention realizes a new approach, i.e., the stable integration of constitutively active growth factor receptor variants and positive selection by growth factor depletion in the cell culture medium, allowing for production in protein-free medium.

The term “selection” as used herein refers to a process of allowing the exclusive survival and proliferation of desired positive cell clones by establishing a selection pressure, i.e., according to the present invention, cultivating said cells in a cell culture medium either not containing the growth factor that is recognized by said growth factor receptor or containing said growth factor at concentrations which are too low to support growth of the cells in the absence of expression of said constitutively active variant of a growth factor receptor, whereas not desired negative cell clones are not able to survive and proliferate under said selection pressure. This use of the term “selection” is to be clearly distinguished from the physical isolation and/or enrichment of desired cells, e.g. by sorting or attachment, out of a larger pool of cells.

The term “producer cell line” as used herein relates to cell lines producing, i.e., expressing, one or more POIs. The term “stable producer cell line” in this respect refers to the fact that the gene(s) encoding said POIs (gene(s) of interest; GOIs), as well as the gene encoding the constitutively active variant of a growth factor receptor, are stably integrated into the cell genome.

In specific embodiments, the cell line provided in step (a) of the methods of the present invention is a mammalian cell line, including a human cell line. Preferably, the cell line is selected from the group consisting of CHO cells, HEK293 cells, CAP cells, Per.C6 cells, BHK cells, Vero cells, MDCK cells, hybridoma cells, and fibroblasts. More preferably, the cell is a CAP cell line.

POIs to be expressed by the cell lines used in the present invention are not particularly limited. They include any proteins expression of which might be desired, for example proteins that are selected from the group consisting of extracellular matrix proteins, growth factors, peptide hormones, cytokines, enzymes, antibodies, antibody fragments, blood clotting factors, protease inhibitors, and viral protein products. Specific examples include human recombinant alpha-1-antitrypsin (rhAAT), fibrinogen, laminins (LAM), interferons (IFN), interleukins (IL), Immunglobulin G (IgG), Immunglobulin M (IgM), bispecific monoclonal antibodies (BsAb), erythropoietin (EPO), Factor VII (FVII), Factor VIII (FVIII), Factor IX (FIX), von-Willebrand-Factor (vWF), C1 esterase inhibitor (C1-inhibitor; C1 Inh), gag-pol from HIV-1, HIV-2, EIAV, SIV, or other retroviridae (gag-pol), rep protein from Adeno-Associated Virus (REP), cap protein from Adeno-Associated Virus (CAP) and variants thereof.

In step (b) of the methods of the present invention, the cell line is stably transfected with (i) a gene encoding a constitutively active variant of a growth factor receptor, wherein growth of said cell line in the absence of expression of said constitutively active variant of a growth factor receptor is dependent on the growth factor that is recognized by said growth factor receptor, and (ii) one or more GOIs encoding said POIs. Respective transfection methods are not particularly limited and are known in the art.

The term “stably transfecting” as used herein indicates the fact that the respective genes are stably integrated into the cell genome.

The term “wherein growth of said cell line in the absence of expression of said constitutively active variant of a growth factor receptor is dependent on the growth factor that is recognized by said growth factor receptor” indicates the fact that in order for the method of the present invention to work as desired, the growth factor receptor/growth factor has to be selected such that growth of the cells under native conditions, i.e., in the untransfected cells, is dependent on said growth factor and respective growth factor receptor signaling.

The term “constitutively active variant of a growth factor receptor” relates to growth factor receptor variants that are in an activated state even in the absence of their respective growth factor ligand.

In a preferred embodiment, the gene encoding a constitutively active variant of a growth factor receptor and the GOIs are present on the same vector. Suitable vectors are not particularly limited and are known in the art.

In specific embodiments, the growth factor receptor and respective growth factors are IGF-1R (insulin-like growth factor 1 receptor), IR (insulin receptor), and IGF-1, IGF-2 and/or insulin; EGFR (epidermal growth factor receptor) and respective EGFR ligands; FGFR (fibroblast growth factor receptor) and FGFs; or PDGFR (platelet-derived growth factor receptor) and PDGFs.

In specific embodiments, the constitutively active variant of a growth factor receptor is an extracellular domain-deleted EGFR, a ZNF198-FGFR1 fusion protein, a PDGFR with point mutations, an IR with point mutations, a constitutively active variant of an extracellular domain-deleted IGF-1R (IGF1R TM-icd) and point mutations thereof to increase activation, as known in the art.

In a particular embodiment, the constitutively active variant of a growth factor receptor is a human CD8-IGF-1R fusion protein. Preferably, said fusion protein comprises or consists of the amino acid sequence of SEQ ID NO: 1. Further, said human CD8-IGF-1R fusion protein is preferably encoded by a nucleic acid comprising or consisting of the nucleotide sequence of SEQ ID NO: 2.

In step (c) of the methods of the present invention, the cell line is cultivated in a cell culture medium either not containing the growth factor that is recognized by said growth factor receptor or containing said growth factor at concentrations which are too low to support growth of the cells in the absence of expression of said constitutively active variant of a growth factor receptor. In this context, respective concentrations which are too low in this respect depend on the specific growth factor and growth factor receptor and can be easily determined by the person skilled in the art. This step is preferably performed for a duration that allows selection to occur, i.e., for a duration that is long enough to allow for the death of cells not expressing the constitutively active variant of the growth factor receptor. In specific examples, step (c) is performed for 9 or 10 passages. In this context, the duration that allows selection to occur is dependent on several factors, including but not limited to the composition of the cell culture medium, the cell line and regulatory elements which are present on the expression vector and may therefore vary depending on these factors. Respective durations can be easily determined by the person skilled in the art.

In a second aspect, the present invention relates to a method for the expression of one or more protein(s) of interest (POIs) in a stable producer cell line, comprising the steps of:
(c) performing the method according to the first aspect of the present invention, as defined above,
(d) expressing said POIs in said stable producer cell line, and
(e) recovering said POIs from the cells or the cell culture medium.

Methods for the expression of POIs in a cell line and the recovery thereof are not particularly limited and are known in the art.

Further, all definitions and limitations defined for the first aspect of the present invention apply to this second aspect of the present invention in an analogous manner.

In a third aspect, the present invention relates to the use of a constitutively active variant of a growth factor receptor as selection marker in the cultivation of cells.

In this aspect, all definitions and limitations defined for the first aspect of the present invention apply to this third aspect of the present invention in an analogous manner. In particular, the cells, growth factor receptors, growth factors, and constitutively active variants of growth factor receptors are as defined above.

The present invention is based on the idea of using constitutively active growth factor receptors (e.g. of IGF-1R) as selection markers. This advantageously adds new, efficient selection markers to the limited panel of currently available selection markers, in particular for mammalian expression systems. Further, the present invention allows for continuous selection pressure during cultivation of producer cells, providing the potential of stabilizing the expression of difficult-to-express target proteins without need of later removal of the selection agent and time-consuming, expensive analytics to prove the absence of the selection agent in the final product. Moreover, the present invention advantageously allows for a cost reduction in large-scale production processes, as there is no need for supplementation of serum-free cell culture medium with the respective growth factors.

The above possibility of establishing a continuous selection pressure, without having to provide additional reagents, is a significant advantage of the methods and uses of the present invention. It allows for the maintenance of high yield expression of recombinant proteins, in particular in cases where a producer cell line is not stably expressing said proteins over several passages. In this context, it should be noted that such unstable producer cell lines are quite common, in particular when protein expression is very high, since excessive protein expression can negatively impact proliferation rates and the cells have a survival and proliferation advantage when shutting down protein production. This silencing effect is advantageously and efficiently countered by establishing a continuous selection pressure according to the present invention.

### THE FIGURES SHOW

FIG. 1:

CAP cell pools stably expressing recombinant human alpha-1-antitrypsin (rhAAT) were generated by transfection of parental CAP cells with either pStbl-CD8-IGF1R-AAT or pStbl-bsd-AAT (control) following selection in IGF-1 depleted or blasticidin containing CAP-CDM growth medium. Mock transfected CAP cells were cultivated in CAP-CDM containing 50 μg/L Long-R3-IGF or in CAP-CDM without IGF over 10 passages as a control. For all pools, cells were grown in 125 mL shake flasks at 185 rpm, 5% CO₂ and 37° C. and were passaged every 72-96 h while adjusting the viable cell density (VCD) to 1×10⁶ cells/mL.

FIG. 2:

RhAAT levels in cell culture supernatants from fed-batch cultures of CAP-bsd-AAT and CAP-CD8-IGF1R-AAT pool cells taken on day 10. RhAAT titers were quantified by ELISA.

The present invention will be further illustrated in the following examples without being limited thereto.

### EXAMPLES

### Example 1

Generation of CAP cells stably expressing human recombinant alpha-1-antitrypsin (AAT) by use of a constitutively active CD8-IGF1R variant as a selection marker.

Introduction

CAP cells are human amniocyte-derived suspension cells which show a growth inhibition in serum-free medium without IGF-1 or insulin supplementation. Thus, they are a suitable cell line for implementation of the new selection marker according to the present invention.

IGF-1R is a transmembrane receptor tyrosine kinase which is essential for cell growth and protein biosynthesis by activating PI3K/Akt-kinase+mTOR and MAP kinase pathways. It is a tetramer comprising two alpha-subunits and two beta-subunits. Its ligands are IGF-1 (highest affinity), IGF-2, and insulin (lowest affinity). Binding of ligands to the alpha-subunit leads to conformational changes (dimerization of alpha-subunits) and subsequent autophosphorylation of specific tyrosine residues of the beta-subunits (Tyr1131, Tyr1135, Tyr1136, Tyr950) followed by binding of receptor substrates (IRS1-4, shc) to their binding sites and initiation of downstream signaling pathways.

Constitutive activation of IGF-1R can be achieved by fusing the intracellular and transmembrane domains thereof to the extracellular domain of human T-cell marker CD8. This method has been previously described in the art, where the CD8-IGF1R fusion construct was utilized as a tool for investigating the role of IGF1R signaling for tumor development in a mouse model. In none of the art, the recombinant expression of CD8-IGF1R and associated constitutive activation of IGF1R served as a selection marker for the generation of stable cell lines.

Alpha-1-antitrypsin (AAT) is a 52 kDa glycoprotein and serine protease inhibitor, targeting in particular neutrophil elastase. Hereditary AAT deficiency leads to severe lung emphysema. Recombinant human AAT (rhAAT) has been successfully produced in CAP cells and is used herein as a model protein for studies involving the CD8-IGF1R as a new selection marker.

### Experimental Procedures

### Cloning of Expression Constructs

Components of the CD8-IGF1R fusion protein are (i) amino acids 1-218 of the human CD8 alpha chain (Uniprot P01732-1), and (ii) amino acids 964-1367 of the intracellular domain of human IGF-1R (NCBI Protein Database NP000866). These are fused in frame without a linker, resulting in the amino acid sequence of CD8-IGF1R fusion protein (SEQ ID NO: 1, as shown hereinafter). The respective cDNA sequence is SEQ ID NO: 2, as shown hereinafter.

cDNA was synthesized by GeneArt (Thermo Fisher Scientific) and subcloned into a pStbl expression vector (CEVEC Pharmaceuticals, Germany) by replacement of the bsd resistance gene by the CD8-IGF1R construct. Components of the final expression construct pStbl-CD8-IGF1R-AAT are (i) cDNA of human alpha1-antitrypsin (AAT) under the control of CMV promoter, (ii) selection cassette containing CD8-IGF1R under the control of human Ubc promoter, (iii) enhancing element for stable transcription of integrated ORFs, (iv) pUC ori for propagation in E. coli, and (v) ampicillin resistance cassette for selection in E. coli.

Verification of the plasmid was by sequencing performed by Eurofins MWG Operon. As control vectors were used (i) pStbl-bsd-AAT (AAT cDNA under control of CMV promoter, bsd selection cassette), and (ii) pStbl-bsd (empty; no GOI, bsd selection cassette).

### Cell Culture

Parental CAP, CAP-bsd-AAT and CAP mock cells were routinely cultivated in chemically defined, serum-free CAP-CDM medium (Merck Millipore, Germany) supplemented with 6 mM L-alanyl-L-glutamine (Biochrom, Germany) and 50 μg/L Long-R3-IGF-I (SAFC, Germany) in shake flasks (125 mL; Corning) on a shaking incubator at 185 rpm (5 cm orbit), 5% CO₂ and 37° C.

CAP-CD8-IGF1R-AAT cells were cultivated in chemically defined, serum-free CAP-CDM medium (Merck Millipore, Germany) supplemented with 6 mM L-alanyl-L-glutamine (Biochrom, Germany) without Long-R3-IGF-I.

During routine cultivation, cells were diluted with fresh medium to a viable cell density of 1×10⁶ cells/ml every 72-96 h. Viable cell density and viability were determined by trypan blue exclusion using a CEDEX XS cell counter (Innovatis, Roche Applied Science).

During fermentation, cells were fed on days 3, 5, and 7 with 10% (v/v) CAP-CDM feed (Merck Millipore, Germany) and 4 mM L-Alanyl-L-glutamine (Biochrom, Germany).

### Nucleofection and Generation of Stable Pools

Stable pools were generated using Lonza's Nucleofector according to the manufacturer's instructions. For each nucleofection reaction, 1×10⁷ cells were harvested by centrifugation (150×g, 5 min). The cells were resuspended in 100 μl complete nucleofector solution V (Lonza) and mixed with 5 μg of the linearized expression vector. The DNA/cell suspension was transferred into a cuvette and the nucleofection was performed using the X001 program. The transfected cells were transferred into 12.5 mL growth medium and cultured as described before at 37° C., 5% CO₂ at 185 rpm.

For generation of stable pools, cells were pelleted by centrifugation and resuspended in selection medium (see Table 1) 72-96 h post-transfection followed by cultivation in a shaking incubator as described before.

CAP mock cells cultivated in IGF-1-depleted CAP-CDM medium served as a negative control.

TABLE 1
Generated CAP cell pools and
corresponding expression vectors and media

| Cell line | Plasmid | Selection medium |
|---|---|---|
| CAP-bsd- | pStbl-bsd-AAT | CAP-CDM + 6 mM L-alanyl-L- |
| AAT | | glutamine + 50 μg/L Long-R3- |
| | | IGF-1 + 5 μg/mL blasticidin |
| CAP-CD8- | pStbl-CD8- | CAP-CDM + 6 mM L-alanyl-L- |
| IGF1R- | IGF1R-AAT | glutamine |
| AAT | | |
| CAP mock | pStbl-bsd (empty) | a) CAP-CDM + 6 mM L-alanyl-L- |
| | | glutamine + 50 μg/L Long-R3-IGF-1 |
| | | b) CAP-CDM + 6 mM L-alanyl-L- |
| | | glutamine |

### ELISA

Concentrations of recombinant AAT in CAP cell culture supernatants were determined using a two-site ELISA. In this microplate-based assay, AAT is captured by an immobilized goat AAT-specific antibody and detected by a second goat AAT-specific antibody which is coupled to horseradish-peroxidase (Bethyl, Cat. #A80-122A/B).

For coating, 96-well microtiter plates were incubated with diluted capture antibody (Bethyl, 1.33 μg/mL in 0.1 M Na₂CO₃/0.1 M NaHCO₃; 100 μL/well) for 1 h at 37° C.

The wells were washed four times with TBS+0.05% (v/v) Tween-20 (=TBST; 200 μl/well) and blocked with TBST+5% (w/v) skim milk powder (=TBSTM; 200 μL/well) overnight at 4° C. After blocking, the plate was washed twice with TBST (200 μL/well), and AAT standard (0.2-200 ng/ml, two-fold serial dilutions in TBST), samples and negative controls were added (100 μL/well). Plates were sealed and incubated for 90 min at 37° C.

Next, the plate was washed four times as described above followed by addition of the detection antibody (66.7 ng/ml in TBSTM; 100 μL/well) and an 1 h incubation step at 37° C. Wells were washed as described above and the TMBD substrate (0.1 mg/ml in 24 mM citrate/52 mM Na₂HPO₄/0.006% H₂O₂, pH 5.0) was added (100 μL/well).

After 10 min incubation at ambient temperature the reaction was stopped by addition of 0.5 M H₂SO₄ (100 L/well) and absorbance at 450 nm (=A450) was measured using a BioRad microtiter plate reader. A standard curve (4-parameter fit) was generated from A450 values of AAT standard dilutions. The curve was used for quantification of recombinant AAT in cell culture supernatants.

### Results

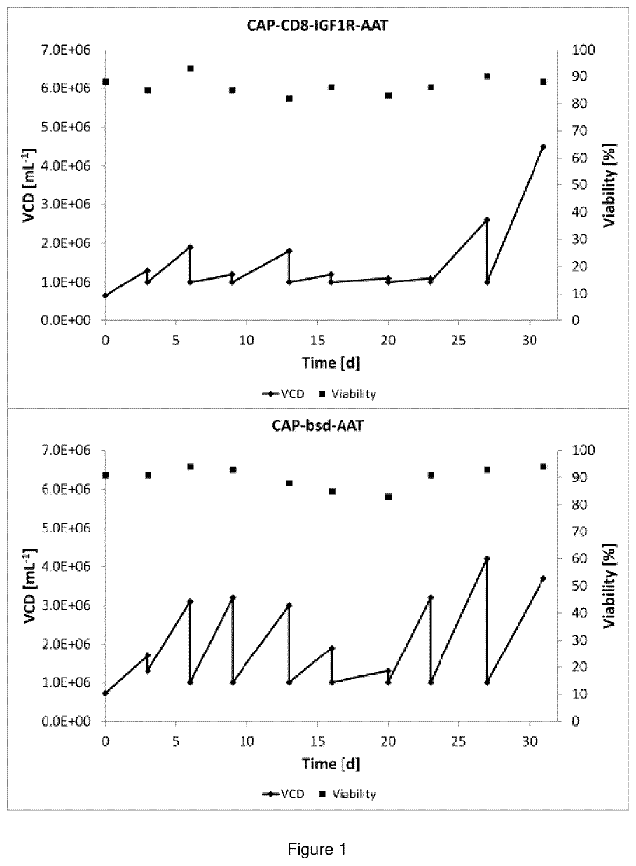

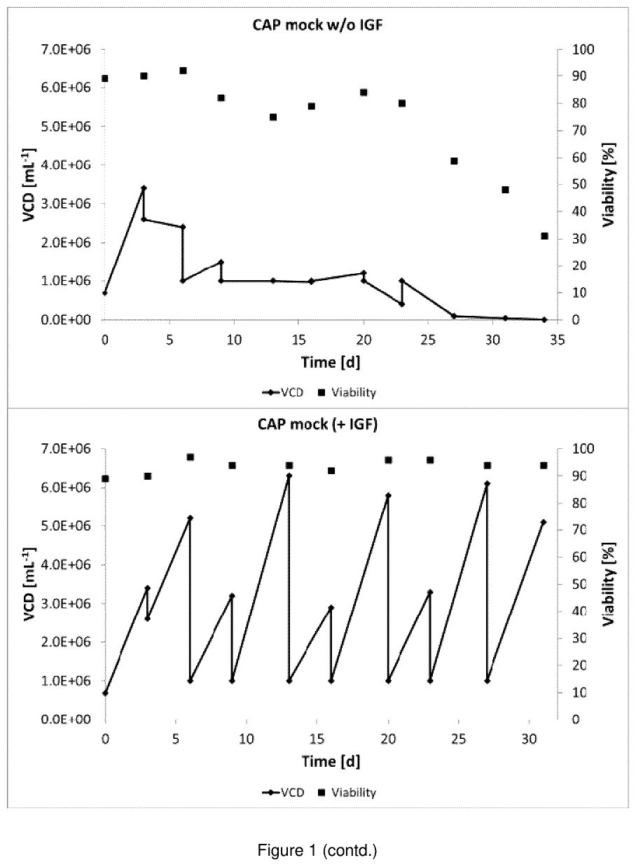

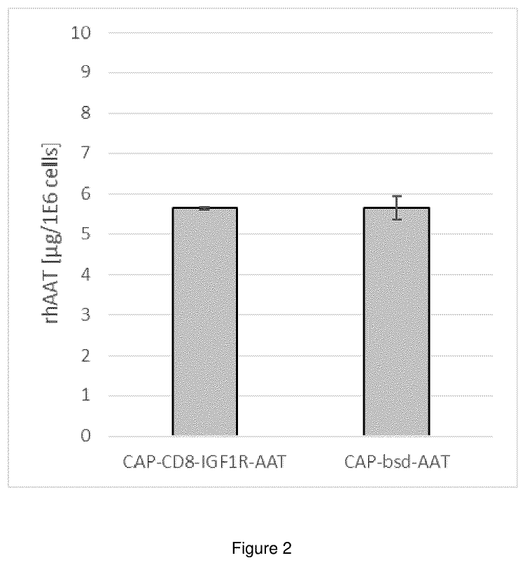

In order to prove the suitability of the constitutively active IGF1R receptor as a selection marker for the generation of stable (CAP) producer cell lines, parental CAP cells were transfected with either the CD8-IGF1R-AAT expression vector or an AAT expression vector containing a blasticidin resistance cassette as a selection marker. CAP cells transfected with the empty pStbl-bsd plasmid (CAP mock) served as a control. One passage after nucleofection, the cells were transferred into the respective selection medium (Table 1) and cultivated over a period of 9 to 10 passages. After 8 to 9 passages in IGF1-depleted growth medium, the CAP-CD8-IGF1R-AAT pool had recovered from selection and reached viable cell densities and viabilities that were comparable to the CAP mock pool which was cultivated in full growth medium containing IGF1 (>2×10⁶ cells/mL, >85% viability after 72-96 h). In contrast, mock transfected CAP cells did not survive cultivation in the IGF-1 depleted growth medium, with viable cell density and viability starting to drop to <2×10⁶ cells/mL and <85% viability after 72-96 h after 3 passages already (FIG. 1). This data indicates that the recombinant expression of CD8-IGF1R in CAP cells indeed leads to constitutive activation of the CD8-IGF1R and related downstream signaling pathways and that this constitutive IGF1R activation can compensate IGF1 starvation and thereby allow for growth of CAP cells in IGF1-depleted cell culture medium.

In order to compare the productivity of a CAP cell pool which was generated by conventional blasticidin selection and a CAP cell pool which was generated using the novel CD8-IGF1R selection marker, a fed-batch production run over 10 days was carried out with the stable CAP cell pools and concentrations of rhAAT in the cell culture supernatants from day 10 were quantified by ELISA. Titers were comparable for both CAP cell pools (5.6 μg AAT/1×10⁶ cells each; FIG. 2), which indicates that the generation of a stable AAT-expressing CAP cell pool by selection using the CD8-IGF1R fusion construct and IGF-1-depleted growth medium within 4 weeks was successful and that the CD8-IGF1R construct is a suitable selection marker for the generation of stable CAP cells when co-transfected with the GOI.

The present invention relates to the following nucleotide sequences.

### SEQ ID NO: 1

### Human CD8-IGF1R Fusion Protein

MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPNSRLGNGVLYASVNPEYFSAADVYVPDEWEVA
REKITMSRELGQGSFGMVYEGVAKGVVKDEPETRVAIKTVNEAASMRERI
EFLNEASVMKEFNCHHVVRLLGVVSQGQPTLVIMELMTRGDLKSYLRSLR
PEMENNPVLAPPSLSKMIQMAGEIADGMAYLNANKFVHRDLAARNCMVAE
DFTVKIGDFGMTRDIYETDYYRKGGKGLLPVRWMSPESLKDGVFTTYSDV
WSFGVVLWEIATLAEQPYQGLSNEQVLRFVMEGGLLDKPDNCPDMLFELM
RMCWQYNPKMRPSFLEIISSIKEEMEPGFREVSFYYSEENKLPEPEELDL
EPENMESVPLDPSASSSSLPLPDRHSGHKAENGPGPGVLVLRASFDERQP
YAHMNGGRKNERALPLPQSSTC

### SEQ ID NO: 2

### cDNA Encoding Human CD8-IGF1R Fusion Protein

(CD8 part (italic), IGF1R part (underlined), Kozak sequence (bold)

GCCACCATGGCCTTACCAGTGACCGCCTTGCTCCTGCCGCTGGCCTTGCT
GCTCCACGCCGCCAGGCCGAGCCAGTTCCGGGTGTCGCCGCTGGATCGGA
CCTGGAACCTGGGCGAGACAGTGGAGCTGAAGTGCCAGGTGCTGCTGTCC
AACCCGACGTCGGGCTGCTCGTGGCTCTTCCAGCCGCGCGGCGCCGCCGC
CAGTCCCACCTTCCTCCTATACCTCTCCCAAAACAAGCCCAAGGCGGCCG
AGGGGCTGGACACCCAGCGGTTCTCGGGCAAGAGGTTGGGGGACACCTTC
GTCCTCACCCTGAGCGACTTCCGCCGAGAGAACGAGGGCTACTATTTCTG
CTCGGCCCTGAGCAACTCCATCATGTACTTCAGCCACTTCGTGCCGGTCT
TCCTGCCAGCGAAGCCCACCACGACGCCAGCGCCGCGACCACCAACACCG
GCGCCCACCATCGCGTCGCAGCCCCTGTCCCTGCGCCCAGAGGCGTGCCG
GCCAGCGGCGGGGGGCGCAGTGCACACGAGGGGGCTGGACTTCGCCTGTG
ATATCTACATCTGGGCGCCCTTGGCCGGGACTTGTGGGGTCCTTCTCCTG
TCACTGGTTATCACCCTTTACTGCAACCACAGGAACCGAAGACGTGTTTG
CAAATGTCCCAACAGCAGGCTGGGGAATGGAGTGCTGTATGCCTCTGTGA
ACCCGGAGTACTTCAGCGCTGCTGATGTGTACGTTCCTGATGAGTGGGAG
GTGGCTCGGGAGAAGATCACCATGAGCCGGGAACTTGGGCAGGGGTCGTT
TGGGATGGTCTATGAAGGAGTTGCCAAGGGTGTGGTGAAAGATGAACCTG
AAACCAGAGTGGCCATTAAAACAGTGAACGAGGCCGCAAGCATGCGTGAG
AGGATTGAGTTTCTCAACGAAGCTTCTGTGATGAAGGAGTTCAATTGTCA
CCATGTGGTGCGATTGCTGGGTGTGGTGTCCCAAGGCCAGCCAACACTGG
TCATCATGGAACTGATGACACGGGGCGATCTCAAAAGTTATCTCCGGTCT
CTGAGGCCAGAAATGGAGAATAATCCAGTCCTAGCACCTCCAAGCCTGAG
CAAGATGATTCAGATGGCCGGAGAGATTGCAGACGGCATGGCATACCTCA
ACGCCAATAAGTTCGTCCACAGAGACCTTGCTGCCCGGAATTGCATGGTA
GCCGAAGATTTCACAGTCAAAATCGGAGATTTTGGTATGACGCGAGATAT
CTATGAGACAGACTATTACCGGAAAGGAGGGAAAGGGCTGCTGCCCGTGC
GCTGGATGTCTCCTGAGTCCCTCAAGGATGGAGTCTTCACCACTTACTCG
GACGTCTGGTCCTTCGGGGTCGTCCTCTGGGAGATCGCCACACTGGCCGA
GCAGCCCTACCAGGGCTTGTCCAACGAGCAAGTCCTTCGCTTCGTCATGG
AGGGCGGCCTTCTGGACAAGCCAGACAACTGTCCTGACATGCTGTTTGAA
CTGATGCGCATGTGCTGGCAGTATAACCCCAAGATGAGGCCTTCCTTCCT
GGAGATCATCAGCAGCATCAAAGAGGAGATGGAGCCTGGCTTCCGGGAGG
TCTCCTTCTACTACAGCGAGGAGAACAAGCTGCCCGAGCCGGAGGAGCTG
GACCTGGAGCCAGAGAACATGGAGAGCGTCCCCCTGGACCCCTCGGCCTC
CTCGTCCTCCCTGCCACTGCCCGACAGACACTCAGGACACAAGGCCGAGA
ACGGCCCCGGCCCTGGGGTGCTGGTCCTCCGCGCCAGCTTCGACGAGAGA
CAGCCTTACGCCCACATGAACGGGGGCCGCAAGAACGAGCGGGCCTTGCC
GCTGCCCCAGTCTTCGACCTGCTGA

1. A cell comprising a polynucleotide expression construct which encodes a constitutively active variant of a growth factor receptor as a selection marker that is expressed in the cell, wherein the variant of a growth factor receptor is a human CD8-IGF-1R fusion protein.

2. The cell of claim 1, wherein the cell is selected from the group consisting of CHO cells, HEK293 cells, CAP cells, Per.C6 cells, BHK cells, Vero cells, MDCK cells, hybridoma cells, and fibroblasts.

3. The cell of claim 1, wherein said human CD8-IGF-1R fusion protein comprises or consists of the amino acid sequence of SEQ ID NO: 1.

4. The cell of claim 1, wherein said human CD8-IGF-1R fusion protein is encoded by a nucleic acid comprising or consisting of the nucleotide sequence of SEQ ID NO: 2.


SEQUENCE LISTING (2 sequences)

SEQ ID NO: 1 - protein, 622 aa, synthetic construct
  REGION 1..622: note = human CD8-IGF1R fusion protein
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 2 - DNA, 1875 bp, synthetic construct
  misc_feature 1..1875: note = cDNA encoding human CD8-IGF1R fusion protein
  source 1..1875: mol_type = other DNA
gccaccatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtcccaacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctga